SEQUENCE LISTING (64 sequences)

SEQ ID NO: 1 - protein, 583 aa, HCV
NGVCWTVYHGAGSKTLAGPKGPITQMYTNVDQDLVGWHRPPGARSLTPCTCGSSDLYLVTRHADVIPVRRRGDSRGSLLSPRPVSYLKGSSGGPLLCPFGHVAGIFRAAVCTRGVAKAVDFIPVETMETTMRSPVFTDNSSPPAVPQTFQVAHLHAPTGSGKSTKVPAAYAAQGYKVLVLNPSVAATLGFGAYMSKAHGVDPNIRTGVRTITTGAPITYSTYGKFLADGGCSGGAYDIIICDECHSTDSTTILGIGTVLDQAETAGARLVVLATATPPGSVTVPHPNIEEVALSNTGEIPFYGKAIPIEAIRGGRHLIFCHSKKKCDELAAKLSSLGLNAVAYYRGLDVSVIPSSGDVVVVATDALMTGFTGDFDSVIDCNTCVTQTVDFSLDPTFTIETTTVPQDAVSRSQRRGRTGRGREGIYRFVTPGERPSGMFDSSVLCECYDAGCAWYELTPAETTVRLRAYLNTPGLPVCQDHLEFWEGVFTGLTHIDAHFLSQTKQAGDNFPYLVAYQATVCAKAQAPPPSWDQMWKCLTRLKPTLQGPTPLLYRLGAVQNEVTLTHPITKYIMTCMSADLEVVT

SEQ ID NO: 2 - protein, 417 aa, HCV
AVDFIPVETMETTMRSPVFTDNSSPPAVPQTFQVAHLHAPTGSGKSTKVPAAYAAQGYKVLVLNPSVAATLGFGAYMSKAHGVDPNIRTGVRTITTGAPITYSTYGKFLADGGCSGGAYDIIICDECHSTDSTTILGIGTVLDQAETAGARLVVLATATPPGSVTVPHPNIEEVALSNTGEIPFYGKAIPIEAIRGGRHLIFCHSKKKCDELAAKLSSLGLNAVAYYRGLDVSVIPSSGDVVVVATDALMTGFTGDFDSVIDCNTAVTQTVDFSLDPTFTIETTTVPQDAVSRSQRRGRTGRGREGIYRFVTPGERPSGMFDSSVLCECYDAGCAWYELTPAETTVRLRAYLNTPGLPVCQDHLEFWEGVFTGLTHIDAHFLSQTKQAGDNFPYLVAYQATVCAKAQAPPPSWDQMW

SEQ ID NO: 3 - protein, 326 aa, HCV
AVDFIPVETMETTMRSPVFTDNSSPPAVPQTFQVAHLHAPTGSGKSTKVPAAYAAQGYKVLVLNPSVAATLGFGAYMSKAHGVDPNIRTGVRTITTGAPITYSTYGKFLADGGCSGGAYDIIICDECHSTDSTTILGIGTVLDQAETAGARLVVLATATPPGSVTVPHPNIEEVALSNTGEIPFYGKAIPIEAIRGGRHLIFCHSKKKCDELAAKLSSLGLNAVAYYRGLDVSVIPSSGDVVVVATDALMTGFTGDFDSVIDCNTAVTQTVDFSLDPTFTIETTTVPQDAVSRSQRRGRTGRGREGIYRFVTPGERPSGMFDSSVL

SEQ ID NO: 4 - protein, 287 aa, HCV
AVDFIPVETMETTMRSPVFTDNSSPPAVPQTFQVAHLHAPTGSGKSTKVPAAYAAQGYKVLVLNPSVAATLGFGAYMSKAHGVDPNIRTGVRTITTGAPITYSTYGKFLADGGCSGGAYDIIICDECHSTDSTTILGIGTVLDQAETAGARLVVLATATPPGSVTVPHPNIEEVALSNTGEIPFYGKAIPIEAIRGGRHLIFCHSKKKCDELAAKLSSLGLNAVAYYRGLDVSVIPSSGDVVVVATDALMTGFTGDFDSVIDCNTAVTQTVDFSLDPTFTIETTTVP

SEQ ID NO: 5 - protein, 236 aa, HCV
APTGSGKSTKVPAAYAAQGYKVLVLNPSVAATLGFGAYMSKAHGVDPNIRTGVRTITTGAPITYSTYGKFLADGGCSGGAYDIIICDECHSTDSTTILGIGTVLDQAETAGARLVVLATATPPGSVTVPHPNIEEVALSNTGEIPFYGKAIPIEAIRGGRHLIFCHSKKKCDELAAKLSSLGLNAVAYYRGLDVSVIPSSGDVVVVATDALMTGFTGDFDSVIDCNTAVTQTVDFS

SEQ ID NO: 6 - protein, 631 aa, HCV
APITAYSQQTRGLLGCIITSLTGRDKNQVEGEVQVVSTATQSFLATCINGVCWTVYHGAGSKTLAGPKGPITQMYTNVDQDLVGWHRPPGARSLTPCTCGSSDLYLVTRHADVIPVRRRGDSRGSLLSPRPVSYLKGSSGGPLLCPFGHVAGIFRAAVCTRGVAKAVDFIPVETMETTMRSPVFTDNSSPPAVPQTFQVAHLHAPTGSGKSTKVPAAYAAQGYKVLVLNPSVAATLGFGAYMSKAHGVDPNIRTGVRTITTGAPITYSTYGKFLADGGCSGGAYDIIICDECHSTDSTTILGIGTVLDQAETAGARLVVLATATPPGSVTVPHPNIEEVALSNTGEIPFYGKAIPIEAIRGGRHLIFCHSKKKCDELAAKLSSLGLNAVAYYRGLDVSVIPSSGDVVVVATDALMTGFTGDFDSVIDCNTCVTQTVDFSLDPTFTIETTTVPQDAVSRSQRRGRTGRGREGIYRFVTPGERPSGMFDSSVLCECYDAGCAWYELTPAETTVRLRAYLNTPGLPVCQDHLEFWEGVFTGLTHIDAHFLSQTKQAGDNFPYLVAYQATVCAKAQAPPPSWDQMWKCLTRLKPTLQGPTPLLYRLGAVQNEVTLTHPITKYIMTCMSADLEVVT

SEQ ID NO: 7 - protein, 41 aa, HCV
IPIEAIRGGRHLIFCHSKKKCDELAAKLSSLGLNAVAYYRG

SEQ ID NO: 8 - protein, 236 aa, HCV
APTGSGKSTKVPAAYAAQGYKVLVLNPSVAATLGFGAYMSKAHGVDPNIRTGVRTITTGAPITYSTYGKFLADGGCSGGAYDIIICDECHSTDSTTILGIGTVLDQAETAGARLVVLATATPPGSVTVPHPNIEEVALSNTGEIPFYGKAIPIEAIRGGRHLIFCHSKKKCDELAAKLSSLGLNAVAYYRGLDVSVIPSSGDVVVVATDALMTGFTGDFDSVIDCNTCVTQTVDFS

SEQ ID NO: 9 - protein, 67 aa, HCV
GKAIPLEVIKGGRHLIFCHSKKKCDELAAKLVALGINAVAYYRGLDVSVIPTSGDVVVVATDALMTG

SEQ ID NO: 10 - protein, 109 aa, Artificial Sequence
  amino acid sequence of the fusion protein
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

SEQ ID NO: 11 - protein, 59 aa, HCV
KPAIIPDREVLYREFDEMEECSQHLPYIEQGMMLAEQFKQKALGLLQTATKQAEAAAPV

SEQ ID NO: 12 - protein, 47 aa, HCV
GGKPALVPDKEVLYQQYDEMEECSQAAPYIEQAQVIAHQFKEKVLGL

SEQ ID NO: 13 - protein, 48 aa, HCV
NDQVVVTPDKEILYEAFDEMEECASKAALIEEGQRMAEMLKSKIQGLL

SEQ ID NO: 14 - protein, 234 aa, HCV
REVLYREFDEMEECSQHLPYIEQGMMLAEQFKQKALGLLQTASRQAEATAPVVQSKWQSLETFWAKHMWNFISGIQYLAGLSTLPGNPAIASLMAFTAAVTSPFSTQQTLLFNILGGWVAAQLAAPSAATSFVGAGIAGAAIGSVGLGKVLVDILAGYGAGVAGALVAFKVMSGEVPTTEDLVNLLPAILSPGALVVGVVCAAILRRHVGPGEGAVQWMNRLIAFASRGNHVSP

SEQ ID NO: 15 - protein, 109 aa, HCV
KPAIIPDREVLYREFDEMEECSQHLPYIEQGMMLAEQFKQKALGLLQTASRQAEATAPVVQSKWQSLETFWAKHMWNFISGIQYLAGLSTLPGNPAIASLMAFTAAVTS

SEQ ID NO: 16 - protein, 80 aa, HCV
MSTNPKPQRKTKRNTDRRPQDVKFPGGGQIVGGVYLLPRRGPRLGVRATRKTSERSQPRGRRQPIPKARRPEGRTWAQPG

SEQ ID NO: 17 - protein, 53 aa, HCV
MSTNPKPQRKTKRNTDRRPQDVKFPGGGQIVGGVYLLPRRGPRLGVRATRKTS

SEQ ID NO: 18 - protein, 48 aa, HCV
MSTNPKPQRKTKRNTDRRPQDVKFPGGGQIVGGVYLLPRRGPRLGVRA

SEQ ID NO: 19 - protein, 15 aa, Artificial Sequence
  linker
GGGGSGGGGSGGGGS

SEQ ID NO: 20 - protein, 4 aa, Artificial Sequence
  linker
GGGG

SEQ ID NO: 21 - protein, 5 aa, Artificial Sequence
  linker
GGGGG

SEQ ID NO: 22 - protein, 6 aa, Artificial Sequence
  linker
GGGGGG

SEQ ID NO: 23 - protein, 7 aa, Artificial Sequence
  linker
GGGGGGG

SEQ ID NO: 24 - protein, 8 aa, Artificial Sequence
  linker
GGGGGGGG

SEQ ID NO: 25 - protein, 9 aa, Artificial Sequence
  linker
GGGGGGGGG

SEQ ID NO: 26 - protein, 10 aa, Artificial Sequence
  linker
GGGGGGGGGG

SEQ ID NO: 27 - protein, 4 aa, Artificial Sequence
  linker
GSGS

SEQ ID NO: 28 - protein, 6 aa, Artificial Sequence
  linker
GSGSGS

SEQ ID NO: 29 - protein, 8 aa, Artificial Sequence
  linker
GSGSGSGS

SEQ ID NO: 30 - protein, 10 aa, Artificial Sequence
  linker
GSGSGSGSGS

SEQ ID NO: 31 - protein, 12 aa, Artificial Sequence
  linker
GSGSGSGSGSGS

SEQ ID NO: 32 - protein, 14 aa, Artificial Sequence
  linker
GSGSGSGSGSGSGS

SEQ ID NO: 33 - protein, 16 aa, Artificial Sequence
  linker
GSGSGSGSGSGSGSGS

SEQ ID NO: 34 - protein, 18 aa, Artificial Sequence
  linker
GSGSGSGSGSGSGSGSGS

SEQ ID NO: 35 - protein, 20 aa, Artificial Sequence
  linker
GSGSGSGSGSGSGSGSGSGS

SEQ ID NO: 36 - protein, 4 aa, Artificial Sequence
  linker
SGSG

SEQ ID NO: 37 - protein, 6 aa, Artificial Sequence
  linker
SGSGSG

SEQ ID NO: 38 - protein, 8 aa, Artificial Sequence
  linker
SGSGSGSG

SEQ ID NO: 39 - protein, 10 aa, Artificial Sequence
  linker
SGSGSGSGSG

SEQ ID NO: 40 - protein, 12 aa, Artificial Sequence
  linker
SGSGSGSGSGSG

SEQ ID NO: 41 - protein, 14 aa, Artificial Sequence
  linker
SGSGSGSGSGSGSG

SEQ ID NO: 42 - protein, 16 aa, Artificial Sequence
  linker
SGSGSGSGSGSGSGSG

SEQ ID NO: 43 - protein, 18 aa, Artificial Sequence
  linker
SGSGSGSGSGSGSGSGSG

SEQ ID NO: 44 - protein, 20 aa, Artificial Sequence
  linker
SGSGSGSGSGSGSGSGSGSG

SEQ ID NO: 45 - protein, 4 aa, Artificial Sequence
  linker
GGGS

SEQ ID NO: 46 - protein, 8 aa, Artificial Sequence
  linker
GGGSGGGS

SEQ ID NO: 47 - protein, 12 aa, Artificial Sequence
  linker
GGGSGGGSGGGS

SEQ ID NO: 48 - protein, 16 aa, Artificial Sequence
  linker
GGGSGGGSGGGSGGGS

SEQ ID NO: 49 - protein, 20 aa, Artificial Sequence
  linker
GGGSGGGSGGGSGGGSGGGS

SEQ ID NO: 50 - protein, 24 aa, Artificial Sequence
  linker
GGGSGGGSGGGSGGGSGGGSGGGS

SEQ ID NO: 51 - protein, 28 aa, Artificial Sequence
  linker
GGGSGGGSGGGSGGGSGGGSGGGSGGGS

SEQ ID NO: 52 - protein, 32 aa, Artificial Sequence
  linker
GGGSGGGSGGGSGGGSGGGSGGGSGGGSGGGS

SEQ ID NO: 53 - protein, 36 aa, Artificial Sequence
  linker
GGGSGGGSGGGSGGGSGGGSGGGSGGGSGGGSGGGS

SEQ ID NO: 54 - protein, 40 aa, Artificial Sequence
  linker
GGGSGGGSGGGSGGGSGGGSGGGSGGGSGGGSGGGSGGGS

SEQ ID NO: 55 - protein, 5 aa, Artificial Sequence
  linker
GGGGS

SEQ ID NO: 56 - protein, 10 aa, Artificial Sequence
  linker
GGGGSGGGGS

SEQ ID NO: 57 - protein, 15 aa, Artificial Sequence
  linker
GGGGSGGGGSGGGGS

SEQ ID NO: 58 - protein, 20 aa, Artificial Sequence
  linker
GGGGSGGGGSGGGGSGGGGS

SEQ ID NO: 59 - protein, 25 aa, Artificial Sequence
  linker
GGGGSGGGGSGGGGSGGGGSGGGGS

SEQ ID NO: 60 - protein, 30 aa, Artificial Sequence
  linker
GGGGSGGGGSGGGGSGGGGSGGGGSGGGGS

SEQ ID NO: 61 - protein, 35 aa, Artificial Sequence
  linker
GGGGSGGGGSGGGGSGGGGSGGGGSGGGGSGGGGS

SEQ ID NO: 62 - protein, 40 aa, Artificial Sequence
  linker
GGGGSGGGGSGGGGSGGGGSGGGGSGGGGSGGGGSGGGGS

SEQ ID NO: 63 - protein, 45 aa, Artificial Sequence
  linker
GGGGSGGGGSGGGGSGGGGSGGGGSGGGGSGGGGSGGGGSGGGGS

SEQ ID NO: 64 - protein, 50 aa, Artificial Sequence
  linker
GGGGSGGGGSGGGGSGGGGSGGGGSGGGGSGGGGSGGGGSGGGGSGGGGS

(19) United States
(12) Patent Application Publication
(10) Pub. No.: US 2023/0331783 A1
(43) Pub. Date: Oct. 19, 2023

(54) HCV Recombinant Antigen and Mutant thereof

(71) Applicant: GUANGDONG FAPON BIOLOGICAL CO., LTD., Dongguan, Guangdong (CN)

(72) Inventors: Peng CUI, Dongguan, Guangdong (CN); Zhiqiang HE, Dongguan, Guangdong (CN); Yuan MENG, Dongguan, Guangdong (CN); Zhongjie WEI, Dongguan, Guangdong (CN); Jing CEN, Dongguan, Guangdong (CN)

(21) Appl. No.: 18/026,603

(22) PCT Filed: Apr. 16, 2021
(86) PCT No.: PCT/CN2021/087907

§ 371 Date: Mar. 16, 2023

(30) Foreign Application Priority Data
Sep. 16, 2020 (CN) 202010976432.1

(51) Int. Cl. C07K 14/005; G01N 33/576; C07K 17/12

(52) U.S. Cl. CPC C07K 14/005; C07K 17/12; G01N 33/5767; C12N 2770/24222; G01N 2333/186; G01N 2469/10; G01N 2469/20

(57) ABSTRACT

The application relates to an HCV recombinant antigen and a mutant thereof. The application provides an HCV recombinant antigen, the HCV recombinant antigen including at least 1 NS3 antigen, wherein at least one cysteine of the NS3 antigen in positions 1192-1517 of an HCV amino acid sequence is mutated into G and/or A, and preferably mutated into A. The application further provides a nucleic acid encoding the HCV recombinant antigen, an expression vector including the nucleic acid, a host cell including the expression vector, a conjugate including the HCV recombinant antigen, and a kit including the HCV recombinant antigen.

### CROSS-REFERENCE TO RELATED APPLICATION

The present application is a National Stage of International Patent Application No. PCT/CN2021/087907, filed on Apr. 16, 2021,and claims priority to Chinese Patent Application No. 202010976432.1 filed to the China National Intellectual Property Administration on Sep. 16, 2020 and entitled “HCV Recombinant Antigen and Mutant thereof”, the disclosure of which is hereby incorporated by reference in its entirety.

### SEQUENCE LISTING

The instant application contains a Sequence Listing which has submitted electronically in ASCII format and is hereby incorporated by reference in its entirety. Said ASCII copy is named PN205670_amended SEQ LlSTINGT.ST25.txt and is 43,563 bytes in size. The sequence listing contains 64 sequences in which the SEQ ID NOs:1-19 are identical in substance to the sequences disclosed in the PCT application, and SEQ ID NOs:20-64 are newly added from the specification of the PCT application. The organism of SEQ ID NOs:10 and 19 are modified from “Artificial” to “Artificial Sequence”, and the notes of SEQ ID NOs:10 and 19 are newly added from the specification of the PCT application. The sequence listing includes no new matter.

### TECHNICAL FIELD

The application relates to the field of Hepatitis C Virus (HCV) detection. Specifically, the application relates to an HCV recombinant antigen and a mutant thereof, which may be used for detecting the presence of an HCV antibody in the sample of a subject. The application further relates to a nucleic acid encoding the HCV recombinant antigen and the mutant thereof, and a related vector, a host cell, an immunoassay method and a detection kit.

### BACKGROUND

Viral hepatitis C, referred to as hepatitis C, is a disease caused by infection with the HCV and is mainly transmitted by blood or body fluids. The World Health Organization estimates that approximately 180 million people worldwide are infected with the hepatitis C, and the global HCV infection rate is about 3%. The prevalence of anti-HCV positivity in our healthy population is 0.7%-3.1 %, about 38 million people. Due to various factors such as the biological characteristics of the virus and host immune functions, it is often difficult for the immunity of a body to effectively clear the virus, leading to the development of chronic hepatitis in about 80% of HCV-infected patients, of which 10%-20% will develop into cirrhosis, and 1%-5% of patients with cirrhosis will develop into hepatocellular carcinoma each year. As a result, the prevalence of chronic HCV infection has become a significant socioeconomic burden in countries around the world.

The HCV is a single-stranded positive-stranded RNA virus. The whole genome is about 9.5 kb long and may be divided into 3 regions, which are a 5′ Untranslated Region (5′UTR), an Open Reading Frame (ORF) and a 3′ Untranslated Region (3′UTR), and the order of the above regions is 5′UTR-C-EI-E2-p7-NS2-NS3-NS4-NS5-3′UTR. The 5′UTR is a highly conserved region, which is an initiation site of viral translation and plays a very important role in an HCV replication process. The ORF includes structural protein regions C, E1 and E2, and non-structural protein regions P7 and NS2-NS5. Envelope regions (E1 and E2) and a core region (C) encode virus particles, and the non-structural protein regions play an important role in virus replication and virus protein synthesis. The core region, and NS2, NS3, NS4A, NS5A and NS5B of the non-structural protein part are important target points of immunodiagnosis research at present.

The gene encoding an HCV core protein is located at nucleotide loci 342-914 in the HCV genome and encodes 191 amino acids, and the amino acid sequence is very conservative. The HCV core protein is a viral protein having various biological functions; and in addition to having a function of assembling the virus particles, the protein plays an important role in HCV proliferation and pathogenesis, and is an important marker of HCV infection. The HCV core protein is produced after HCV infection and before the positive conversion of antibodies in the body, and is an important indicator for reflecting HCV replication and HCV viral load in patients. Since the protein is highly conserved and highly immunogenic, the protein is widely used in HCV diagnostics and vaccine studies. The NS3 gene is located at nucleotide loci 3300-5200 of the HCV whole genome sequence and encodes 630 amino acids. The NS3 protein has a protease function. 189 amino acids on an N terminal have serine protease activity, and 442 amino acids on a C terminal have the activity of nucleoside triphosphatase (NTPase) and RNA helicase. During HCV infection, anti-NS3 antibodies are the first to appear. The NS3 protein is highly antigenic, and almost all HCV-infected patients produce high-titer and specific anti-NS3 antibodies. The NS4 gene is located at nucleotides 4974-5133 of the whole HCV genome and encodes two proteins NS4A and NS4B. NS4A is a short peptide of 54 amino acids; and NS4B is a 27 kDa-sized transmembrane protein localized to an ER membrane and has strong hydrophobicity. The NS4 region includes at least two immunodominant sequence sites and is highly antigenic, and the vast majority of HCV-infected patients produce antibodies against the region. The NS5 gene coding region is 3117 nucleotides long, encodes two proteins NS5A and NA5B, and is the longest coding region in the HCV genome.

With the development of a genetic engineering technology, the molecular biology of HCV viral genes has become increasingly mature and been applied to diagnostic reagents. An HCV diagnostic reagent mainly uses gene fragments, such as core, NS3 and NS4, on different detection platforms. In the diagnostic reagent, HCV diagnostic raw materials are mostly in the mode of individual HCV-core, individual NS3, chimeric core+NS3, chimeric NS3+core, and chimeric NS3+NS4. The adjustment of a chimeric mode can improve sensitivity to a certain extent, but is not very helpful in the specificity. Therefore, there is still a need in the art for high sensitivity and high specificity products for detecting HCV.

### SUMMARY

The application provides an HCV recombinant antigen. The HCV recombinant antigen includes at least one NS3 antigen, wherein at least one cysteine in the NS3 antigen is mutated into G or A, preferably mutated into A; and the mutation site is located between the HCV amino acid positions 1192 and 1517.

The application provides an HCV recombinant antigen. The HCV recombinant antigen includes at least one NS3 antigen, wherein at least one cysteine of the NS3 antigen at position 1305/1315/1318/1394/1400/1454 of a corresponding HCV amino acid sequence is mutated into G or A, and preferably mutated into A.

In one or more implementations, the specificity of the HCV recombinant antigen for detecting an HCV antibody is not less than 98.0%, for example, not less than 98.2%, not less than 99.0%, not less than 99.6%, not less than 99.8% and not less than 99.9%.

In one or more implementations, the NS3 antigen includes NS3 polypeptide selected from positions 1027-1657 of the HCV amino acid sequence, for example, the length of the polypeptide is 41-631 amino acids, for example, 41, 50, 60, 70, 80, 90, 100, 150, 200, 250, 300, 350, 400, 450, 500, 550 or 600 amino acids; for example, the NS3 polypeptide includes positions 1027-1657, 1380-1420, 1075-1657, 1230-1465, 1192-1478, 1377-1443, 1192-1608 or 1192-1517 of the HCV amino acid sequence; and for example, the NS3 polypeptide (unmutated polypeptide) includes sequences selected from SEQ ID NO:1-9.

In one or more implementations, the HCV recombinant antigen further includes another HCV antigen, for example, another one or more (for example, at least one, two, three or more) NS3 antigens, another one or more (for example, at least one, two, three or more) NS4 antigens, another one or more (for example, at least one, two, three or more) NS5 antigens, and/or another one or more (for example, at least one, two, three or more) core antigens.

In one or more implementations, the NS3 antigen, the NS4 antigen, the NS5 antigen and/or the core antigen are directly connected or connected optionally by means of one or more linkers, for example, the linker is (G)n, (GS)n, (SG)n, (GGGS)n or (GGGGS)n, wherein n is one, two, three, four, five, six, seven, eight, nine, tenor a larger integer.

The linker includes but is not limited to:

GGGG(SEQ ID NO:20)

GGGGG(SEQ ID NO:21);

GGGGGG(SEQ ID NO:22);

GGGGGGG(SEQ ID NO:23);

GGGGGGGG(SEQ ID NO:24);

GGGGGGGGG(SEQ ID NO:25);

GGGGGGGGGG(SEQ ID NO:26);

GSGS(SEQ ID NO:27);

GSGSGS(SEQ ID NO:28);

GSGSGSGS(SEQ ID NO:29);

GSGSGSGSGS(SEQ ID NO:30);

GSGSGSGSGSGS(SEQ ID NO:31);

GSGSGSGSGSGSGS(SEQ ID NO:32);

GSGSGSGSGSGSGSGS(SEQ ID NO:33);

GSGSGSGSGSGSGSGSGS(SEQ ID NO:34);

GSGSGSGSGSGSGSGSGSGS(SEQ ID NO:35);

SGSG(SEQ ID NO:36);

SGSGSG(SEQ ID NO:37);

SGSGSGSG(SEQ ID NO:38);

SGSGSGSGSG(SEQ ID NO:39);

SGSGSGSGSGSG(SEQ ID NO:40);

SGSGSGSGSGSGSG(SEQ ID NO:41);

SGSGSGSGSGSGSGSG(SEQ ID NO:42);

SGSGSGSGSGSGSGSGSG(SEQ ID NO:43);

SGSGSGSGSGSGSGSGSGSG(SEQ ID NO:44);

GGGS(SEQ ID NO:45);

GGGSGGGS(SEQ ID NO:46);

GGGSGGGSGGGS(SEQ ID NO:47);

GGGSGGGSGGGSGGGS(SEQ ID NO:48);

GGGSGGGSGGGSGGGSGGGS(SEQ ID NO:49);

GGGSGGGSGGGSGGGSGGGSGGGS(SEQ ID NO:50);

GGGSGGGSGGGSGGGSGGGSGGGSGGGS(SEQ ID NO:51);

GGGSGGGSGGGSGGGSGGGSGGGSGGGSGGGS(SEQ ID NO:52);

GGGSGGGSGGGSGGGSGGGSGGGSGGGSGGGSGGGS(SEQ ID NO:53)
;

GGGSGGGSGGGSGGGSGGGSGGGSGGGSGGGSGGGSGGGS(SEQ ID NO
:54);

GGGGS(SEQ ID NO:55);

GGGGSGGGGS(SEQ ID NO:56);

GGGGSGGGGSGGGGS(SEQ ID NO:57);

GGGGSGGGGSGGGGSGGGGS(SEQ ID NO:58);

GGGGSGGGGSGGGGSGGGGSGGGGS(SEQ ID NO:59);

GGGGSGGGGSGGGGSGGGGSGGGGSGGGGS(SEQ ID NO:60);

GGGGSGGGGSGGGGSGGGGSGGGGSGGGGSGGGGS(SEQ ID NO:61);

GGGGSGGGGSGGGGSGGGGSGGGGSGGGGSGGGGSGGGGS(SEQ ID NO
:62);

GGGGSGGGGSGGGGSGGGGSGGGGSGGGGSGGGGSGGGGSGGGGS(SEQ
ID NO:63);

or

GGGGSGGGGSGGGGSGGGGSGGGGSGGGGSGGGGSGGGGSGGGGSGGGGS
(SEQ IDNO:64).

In one or more implementations, the HCV recombinant antigen includes N- and/or C-terminal modifications, for example, histidine tags, biotinylation and/or detectable marker modifications.

In one or more implementations, HCV includes HCV genotype 1a, 1b, 2a, 2b, 2c, 2d, 3a, 3b, 3c, 3d, 3e, 3f, 4a, 4b, 4c, 4d, 4e, 4f, 4g,4h, 4i, 4j, 5a or 6a.

In one or more implementations, the NS3 antigen has cysteine mutations at positions 1305, 1315, 1318, 1394, 1400 and 1454 of the corresponding HCV amino acid sequence; the cysteine mutation at each position independently selects the mutation from cysteine to G or the mutation from the cysteine to A; and preferably, the cysteine mutation at each of the position is from the cysteine to A.

The application further provides a nucleic acid encoding the HCV recombinant antigen described here.

The application further provides an expression vector of the nucleic acid described here.

The application further provides a host cell of the expression vector described here, for example, an Escherichia coli cell.

The application further provides a conjugate, which includes the HCV recombinant antigen described here.

In one or more implementations, the conjugate further includes a solid support, a detectable marker or a binding partner which is conjugated with the HCV recombinant antigen. For example, the HCV recombinant antigen in the conjugate may be directly or indirectly conjugated. For example, the solid support includes magnetic particles, a microtiter plate or a cellulose membrane. For example, the detectable marker may be a metal particle, a fluorescent marker, a chromophore marker, an electron-dense marker, a chemiluminescent marker, a radioactive marker, or an enzyme marker; and for example, the detectable marker may be colloidal gold, radio isotope, fluorophores, a spin marker, or a bacteriophage marker; for example, the detectable marker may be rhodamine, fluorescein, acridinium ester, luciferase, horseradish peroxidase, alkaline phosphatase, β-galactosidase, glucoamylase, lysozyme, carbohydrate oxidase, glucose oxidase, galactose oxidase or a glucose-6-phosphate dehydrogenase marker. For example, the binding partner includes biotin, streptavidin or avidin.

The application further provides a kit, which includes the HCV recombinant antigen described here or the conjugate described here. In some implementations, the kit may include another HCV antigen, and the HCV antigen includes epitope that is immunoreactive with an HCV antibody. In some implementations, the HCV recombinant antigen described in the application and the HCV antigen may be co-encapsulated on the same solid phase or separately encapsulated on separate solid phases.

In one or more implementations, the kit further includes a detection antibody. In some implementations, the detection antibody may be an antibody that detects human antibodies. In some implementations, the kit may include an anti-HCV antibody. In some implementations, the detection antibody and/or the anti-HCV antibody may be marked with a detectable marker.

The application further provides applications of the HCV recombinant antigen described here or the conjugate described here in preparation of a kit for detecting an HCV antibody or antigen from a sample of a subject.

The application further provides applications of the HCV recombinant antigen described here or the conjugate described here for detecting an HCV antibody or antigen from a sample of a subject.

The application further provides a method for detection an HCV antibody or antigen in a sample of a subject. The method includes contacting the HCV recombinant antigen described here or the conjugate described here with a sample of a subject.

The application further provides a method for diagnosing hepatitis C. The method includes the following operations.
The HCV recombinant antigen described here or the conjugate described here is used to detect an HCV antibody or antigen from a sample of a subject.
A detection result is analyzed.

In one or more implementations, the sample includes biological tissue, cells or body fluids in a healthy or pathological state, such as blood samples, for example, plasma, serum or blood products, such as semen or vaginal secretions.

### DETAILED DESCRIPTION OF THE EMBODIMENTS

In order to make the purposes, technical solutions and advantages of the implementations of the application clearer, the technical solutions in the implementations of the application will be clearly and completely described below. If specific conditions are not indicated in the implementations, the implementations are carried out in accordance with the conventional conditions or the conditions recommended by manufacturers. Reagents or instruments used are conventional products that may be purchased commercially if the manufacturers are not specified.

During an in-depth study of construction of a recombinant HCV antigen, the inventor found that at least one of cysteine in an existing NS3 region at position 1305/1315/1318/1394/1400/1454 is mutated into G/A (preferably mutated into A), which has an unexpectedly superior effect, including, for example, improved specificity of HCV antibody detection in the sample of the subject, thereby reducing false positive rates.

In some implementations, the method and/or kit described here may be used for detecting the presence of the HCV antibody in the sample of the subject, measuring the volume or concentration of the HCV antibody, monitoring progression of diseases, monitoring a treatment effect, and/or determining the onset of hepatitis in subjects or onset risks.

The method and/or products in the application solve one or more problems of low sensitivity and low specificity. Therefore, compared with existing methods, the method of the application has the following one or more advantages: sensitivity is improved, and/or specificity is improved.

In one or more implementations, the specificity of the HCV recombinant antigen for detecting an HCV antibody is not less than 98.0%, for example, not less than 98.1%, not less than 98.2%, not less than 98.3%, not less than 98.4%, not less than 98.5%, not less than 98.6%, not less than 98.7%, not less than 98.8%, not less than 98.9%, not less than 99.0%, not less than 99.1%, not less than 99.2%, not less than 99.3%, not less than 99.4%, not less than 99.5%, not less than 99.6%, not less than 99.7%, not less than 99.8% and not less than 99.9%.

The recombinant HCV antigen of the application may be prepared by means of any appropriate method known in the art. For example, in some implementations, a nucleic acid encoding the recombinant HCV antigen of the application may be prepared, and cloned to any appropriate vector such as plasmid, phage and cosmid; and then recombinant molecules are expressed by means of an appropriate expression system or a host (for example, insect, mammalian, bacterial, viral, and yeast expression systems). In some implementations, host cells suitable for recombinant expression are well known in the art, including, but not limited to: animal cells, such as Chinese Hamster Ovary (CHO) cells, HeLa cells and human embryonic kidney cells; bacterial host cells, such as escherichia coli, bacillus subtilis and streptococcus cells; and yeast host cells, such as saccharomyces cerevisiae. In some implementations, the recombinant HCV antigen may be prepared by using one or more linkers to connect antigen fragments. The linker used here includes a natural or artificial polypeptide sequence that connects target polypeptide sequences. The linker may have a length of about 4-50 amino acids, for example, a length of about 6-30 amino acids.

In some implementations, the genotype of HCV in the application is not specifically limited, and may include, for example, HCV genotype 1a, 1b, 2a, 2b, 2c, 2d, 3a, 3b, 3c, 3d, 3e, 3f, 4a, 4b, 4c, 4d, 4e, 4f, 4 g,4h, 4i, 4j, 5a or 6a. The NS3 antigen in the application being at position 1305/1315/1318/1394/1400/1454 of the corresponding HCV amino acid sequence should be understood as corresponding to the position where the cysteine is located in the embodiments of the application.

In some implementations, in addition to including the mutated NS3 antigen described here, the recombinant HCV antigen of the present application may further include another HCV antigen, for example, another 1 or more (for example, at least 1, 2, 3 or more) NS3 antigens, another 1 or more (for example, at least 1, 2, 3 or more) NS4 antigens, another 1 or more (for example, at least 1, 2, 3 or more) NS5 antigens, and/or another 1 or more (for example, at least 1, 2, 3 or more) core antigens. In some implementations, if the recombinant HCV antigen includes many (for example, 2, 3 or more) antigens of the same type, for example, many NS3, NS4, NS5 and core antigens, the antigens may be the same or different.

In some implementations, in the mutated HCV NS3 antigen, there are mutations at one or more of positions 1305, 1315, 1318, 1394, 1400 and 1454 of the corresponding HCV amino acid sequence, for example, cysteine (C) is mutated into alanine (A) or glycine (G), preferably, the cysteine (C) is mutated into the alanine (A).

As used in the description of mutation, an amino acid abbreviation A stands for alanine; an abbreviation G stands for glycine; an abbreviation C stands for cysteine; and an abbreviation S stands for serine.

In some implementations, in the mutated HCV NS3 antigen, there are mutations at positions 1305, 1315, 1318, 1394, 1400 and 1454 of the corresponding HCV amino acid sequence; and preferably, each mutation is independently selected from a mutation from C to A or from a mutation from C to G. In some implementations, in the recombinant HCV antigen, all Cs at positions 1305, 1315, 1318, 1394, 1400 and 1454 of the corresponding HCV amino acid sequence are mutated into A.

In some implementations, the recombinant HCV antigen of the application may include further modifications, for example, label modifications. The recombinant HCV antigen of the application is not particularly limited, for example, may be a protein purification label, for example, an affinity label, such as a biotin label. As is well known in the art, separation of target HCV antibodies may be achieved by specifically binding the target HCV antibodies and labeled recombinant HCV antigens and performing separation by identifying a labeled binding partner.

In some implementations, the HCV antibody in the sample of the subject identifies epitope in the recombinant HCV antigen of the application, such that the recombinant HCV antigen of the application may be used in immunoassays to detect the HCV antibody in the sample of the subject. In some implementations, the recombinant HCV antigen of the application may be used for immunoassays, such as ELISA, fluorescence immunochromatography, colloidal gold immunochromatography, chemiluminescence assay, electrochemiluminescence assay, Indirect Immunofluorescence Assay (IFA), Radioimmunoassay (RIA) and other non-enzyme-linked antibody binding tests or methods. In some implementations, the recombinant HCV antigen of the application may be used as both a capture antigen and a detection antigen, or is used as the detection antigen and the capture antigen at the same time. In some implementations, another antigen paired with the recombinant HCV antigen of the application may be the same or different, as long as each fragment in the HCV recombinant antigen of the application is included. For example, in some implementations, another antigen paired with the recombinant HCV antigen of the application may be the identical antigen as the recombinant HCV antigen of the application, or may be a different antigen including corresponding fragments of the recombinant HCV antigen of the application. In some implementations, for example, in an ELISA solution, the recombinant HCV antigen may be used as the capture antigen to encapsulate a solid phase such as magnetic beads, so as to capture the HCV antibody in the sample, and then a result is read after color development. In some implementations, antigens or antibodies used in immunoassays may be fixed on a surface, for example, on a solid support, such as plastic, a membrane such as a nitrocellulose membrane, glass, a magnetic bead or a metal support. In some implementations, the sample of the subject is in contact with the solid support, and then is in contact with the detection antibody or the detection antigen with a detectable marker for color development after identification. Herein, the sample of the subject may include biological tissue, cells or body fluids in a healthy or pathological state, such as blood samples, for example, plasma, serum or blood products, such as semen or vaginal secretions.

In some implementations, the detection antigen or the detection antibody (for example, an anti-human IgG antibody or an anti-human IgM antibody) may be marked with the detectable marker. In some implementations, the detectable marker used for marking the antigens or the antibodies is not particularly limited. In some implementations, the marker may include, but not limited to, a fluorescent marker, a chromophore marker, an electron-dense marker, a chemiluminescent marker, a radioactive marker, and an indirect marker such as enzyme or ligand, for example, indirect detection is achieved by means of enzymatic reaction or molecular interaction. In some implementations, exemplary markers include, but are not limited to, radioisotopes, fluorophores, rhodamine and derivatives thereof, luciferase, fluorescein, Horseradish Peroxidase (HRP), alkaline phosphatase, β-galactosidase, glucoamylase, lysozyme, carbohydrate oxidase such as glucose oxidase, galactose oxidase and glucose-6-phosphate dehydrogenase, biotin/avidin, spin markers, bacteriophage markers, and the like.

In some implementations, the application provides a method, for example, immunoassay for detecting the presence of an anti-HCV antibody in a sample of a subject. The method may include: contacting the recombinant HCV antigen of the application with the sample; insofar as there is the HCV antibody in the sample, forming a complex of the HCV antibody and the recombinant HCV antigen; and detecting the presence of the complex, wherein the presence of the complex indicates that there is the HCV antibody in the sample. In some implementations, the complex may be detected by determining the detectable marker (for example, the fluorescent marker). In some implementations, the sample containing or suspected to contain the HCV antibody may be in contact with the recombinant HCV antigen of the application and at least one detection antibody (for example, a second detection antibody or a third detection antibody, for example, an anti-IgG antibody or an anti-lgM antibody with the detectable marker) at the same time or in any order. In some implementations, the method and/or kit of the application is applicable to any appropriate automatic or semi-automatic system.

In some implementations, the application provides a kit containing the recombinant HCV antigen of the application, for example, a kit used for detecting the presence of an HCV antibody in a sample of a subject. In some implementations, the kit includes a reagent suitable for performing immunoassay. In some implementations, the kit may include a specification for the use of an immunodiagnostic reagent (for example, a conjugate containing a recombinant HCV antigen) of the application in the immunoassay for detecting the HCV antibody. In some implementations, the kit may include a calibrator or a control, for example, a standard or control HCV antibody. In some implementations, the recombinant HCV antigen or conjugate of the application is contained on a container, such as a test tube, a microplate or a test strip, in the kit. In some implementations, the kit may further include a solid support, such as a magnetic bead, a test tube, a microplate, a cuvette, a thin film, a filter paper, an injection syringe, a pipette, a buffer solution such as a determination buffer solution, a washing buffer solution, a pretreatment reagent, a substrate solution with the detectable marker such as an enzyme marker.

In some implementations, the application includes a test strip containing the recombinant HCV antigen, for example, a lateral chromatography test strip. In some implementations, the test strip contains the recombinant HCV antigen encapsulated on the solid phase, at least one detection antibody with the detectable marker (such as colloidal gold) or at least one detection antigen. In some implementations, the test strip contains the recombinant HCV antigen with the detectable marker, at least one detection antibody encapsulated on the solid phase or at least one detection antigen. In some implementations, by means of the application, the HCV antibody in the subject may be rapidly and accurately detected by means of visual observation or a fully-automated chemiluminescent instrument. In some implementations, the kit is based on double antigen sandwich immunoassay. For example, in some implementations, the antibody in the sample is captured by the recombinant HCV antigen encapsulated on the solid phase; or the antibody in the sample is detected by the recombinant HCV antigen which is marked by the detectable marker. In some implementations, the kit is based on indirect immunoassay. For example, in some implementations, the antibody in the sample is captured by the recombinant HCV antigen encapsulated on the solid phase. In some implementations, an excitation solution is added; a luminous value is measured by the fully-automated chemiluminescent instrument; the luminous value is positively correlated with the total concentration of the antibody in the sample; and negative and positive are determined by comparing the luminous value with a critical value.

The application further provides applications of the HCV recombinant antigen described here or the conjugate described here for detecting an HCV antibody or antigen from a sample of a subject.

The application further provides a method for detection an HCV antibody or antigen in a sample of a subject. The method includes contacting the HCV recombinant antigen described here or the conjugate described here with a sample of a subject.

The application further provides a method for diagnosing hepatitis C. The method includes the following operations.
The HCV recombinant antigen described here or the conjugate described here is used to detect an HCV antibody or antigen from a sample of a subject.
A detection result is analyzed.

The term antibody used here, such as the detection antibody, is not particularly limited, may include, for example, a monoclonal antibody, a polyclonal antibody, a multi-specific antibody, a human antibody, a humanized antibody, a recombinant antibody, a chimeric antibody, a single-chain antibody and a single-domain antibody, or may include functional fragments of the antibody such as a Fab fragment, a F(ab′) fragment, a Fab′-SH fragment, a F(ab′)2 fragment, a Fd fragment, a Fv fragment, a single-chain Fv fragment (scFv), a dAb fragment and an isolated Complementary Decision Region (CDR), an anti-idiotypic antibody, a bifunctional antibody, a double-domain antibody, or the like.

The term fusion protein used here refers to a protein or polypeptide that is expressed in fusion with the HCV antigen. The protein or the polypeptide may be used for affinity or marking (directly or indirectly). For example, in some implementations, the fusion protein is expressed in fusion with the HCV antigen, and the marking of the marker (such as colloidal gold) is achieved by means of specific binding between the anti-fusion protein antibody and the fusion protein.

### Embodiment 1 Construction of a Vector Containing a Fusion Protein Encoding Sequence

In this embodiment, a fusion protein was selected as a specific protein for clone construction, and a sequence of the fusion protein is shown as follows; a coding sequence of the fusion protein may be obtained by means of E.coli, PET32A and gene synthesis; and at the same time, codon optimization may be performed to reach an optimal expression state. In this embodiment, the following sequence was used to construct an expression vector containing a HIS tag, (GGGGS)₃ at the 3′ end of the fusion protein sequence was introduced, and a BgIII/EcoRI endonuclease site was reserved,

An amino acid sequence of the fusion protein (SEQ ID NO:1 0):

MSDKIIHLTDDSFDTDVLKADGAILVDFVVAEVVCGPCKMIAPILDE
IADEYQGKLTVAKLNIDQNPGTAPKYGIRGIPTLLLFKNGEVAATKVGAL
SKGQLKEFLDANLA

### Embodiment 2 Construction of Recombinant Protein Expression Plasmids for HCV Genetic Engineering

Gene segments (referring to Embodiment 5) of an HCV recombinant protein are designed; and the gene fragments may be spliced by means of restriction endonuclease and a T4 DNA ligase tool enzyme in a manner of enzyme digestion and connection, or may be spliced in the form of primer pouches by means of bridge PCR. Sections may be connected by means of or without linkers, and the linkers may use the form of common linkers, for example, GGGGSGGGGSGGGGS (SEQ ID NO:19). The above gene fragments were constructed onto the expression vector of Embodiment 1 by means of the restriction endonuclease and the T4 DNA ligase tool enzyme in the manner of enzyme digestion and connection.

### Embodiment 3 HCV Recombinant Protein Induction Expression and Purification

Recombinant protein induction expression: the constructed expression plasmids were transformed into E. coli BL21 competent cells (New England Biolabs (NEB), article number: C2530H) by means of a thermal stimulation method, coated on an LB plate containing 50 µ g/ml Kan, and was cultured for 16 h at 37° C.Single colonies were selected, and positive strains identified by bacterial solution PCR and double digestion were selected for conservation and inoculated in an LB culture medium containing 50 µg/ml Kan, and shaking culture was performed at 37° C.; after OD600 reached 0.6-0.8, 1.0 mM IPTG was added, and culture was performed for 2-4 h at 37° C., so as to induce protein expression; the total protein was extracted, and the expression of the recombinant protein was identified by means of SDS-PAGE; and by means of a 6 x HIS tag on the N terminal of the recombinant protein, the protein with the purity being 96% was obtained after nickel ion chelation purification and SP column purification.

### Embodiment 4 Evaluation of the Specificity of a Double Antigen Sandwich Assay for HCV Recombinant Protein for Detection of Hepatitis C Antibodies

The purified HCV recombinant antigen was prepared into an HCV colloidal gold lateral chromatography test strip. A specific process included the following:

### 4.1 Colloidal Gold Preparation

100 ml of ultrapure water was added to a conical flask, and then was heated to boiling on a magnetic heating stirrer; a 1 ml of a 1% chloroauric acid (Sigma-Aldrich, article number: 16961-25-4) solution was added, 1 ml of 1% sodium citrate (Sigma-Aldrich, article number: 6132-04-3) aqueous solution was immediately added after boiling; continuous boiling was kept for 10 minutes; and then natural cooling was performed.

### 4.2 Preparation of an HCV Recombinant Antigen-Colloidal Gold Conjugate

10 ml of the colloidal gold was taken and placed into a beaker, 170 µl of 0.2 M K₂CO₃was added during stirring, so as to adjust pH to 7.5, and stirring was continuously performed for 20 seconds; a certain quantity of anti-fusion protein monoclonal antibodies was added, and stirring was continuously performed for 10 minutes; 0.1 ml of 10%BSA was added, and stirring was continuously performed for 5 minutes; centrifugation was performed on 8000 g of the mixture for 20 minutes, supernatant was discarded, and precipitation was adjusted to 1 ml bycolloidal gold diluent(20 mM PB, 150 mM NaCl, 1%BSA, 0.2%TritonX-100, 2%Sucrose, 0.01%Proclin300); and finally, a certain quantity of HCV recombinant antigen with the fusion protein was added to 1 ml of an anti-fusion protein monoclonal antibody complex marked with the colloidal gold, and was stored at -4° C. after well mixing, and the HCV recombinant antigen-colloidal gold conjugate was prepared.

### 4.3 Preparation of a Gold-Marked Pad

The HCV recombinant antigen-colloidal gold conjugate was diluted 10 folds with the colloidal gold diluent, then glass fiber (Watman) is immersed, freeze-drying was performed, and the gold-marked pad was prepared.

### 4.4 Nitrocellulose Membrane (NC Membrane) Encapsulation

A corresponding HCV recombinant antigen (which is the same as the recombinant antigen used for detection of the same group) was used as an HCV encapsulated antigen; the HCV encapsulated antigen was diluted to 0.5 mg/ml with a detection line diluent (10 mM PBS+2% sucrose), so as to prepare into a detection line working solution; and the corresponding position of an NC membrane (Millipore, article number:HF135002) was scribed with the detection line working solution by using a membrane dotting instrument, and drying was performed for 1 hour at 37° C.

### 4.5 Assembly

The gold-marked pads was respectively assembled with the encapsulated NC membranes, absorbent papers, polyester plates and sample pads, so as to form HCV gold-marked detection reagent strips.

### 4.6 Detection Method

80 µl of a sample to be detected (for example, serum) was added to the sample pad, and placed for 15 minutes at room temperature, and then a result was determined.

### 4.7 Performance Evaluation of the HCV Colloidal Gold Test Strip

Specificity detection was performed by using 1000 HCV antibody-negative samples. The specificity shown as a percentage herein was obtained by testing 1000 known negative samples using the above HCV colloidal gold test strips, and was calculated as follows:

$$\text{Specificity} = \left[\text{TN} / \left(\text{TN+FP}\right)\right] \times 100 \%$$

Where TN is the number of samples with negative detection results, and FP is the number of samples with positive (that is, false positive) detection results.

With regard to this embodiment, the specificity is the number of samples with negative detection results divided by 1000 and multiplied by 100%.

### Embodiment 5

Construction of an NS3 recombinant antigen was performed according to the following; the specificity of the NS3 recombinant antigen was detected; and results showed that by mutating at least one cysteine of the NS3 antigen at 1305/1315/1318/1394/1400/1454 of an NS3 region into G and/or A, the specificity NS3 detection can be improved; and preferably, the cysteine is mutated into A.

The constructed sections of the NS3 recombinant antigen, included mutation and detection specificity are described in detail below.

NS3-10 recombinant antigen, corresponding to HCV amino acid sequence position: 1075-1657, detection specificity: 98.4%.

NS3-11 recombinant antigen, mutations included on the NS3-10 sequence: C1305A, C1315A, C1318A, C1394A, C1400A, and C1454A, detection specificity: 99.9%.

NS3-20 recombinant antigen, corresponding to the HCV amino acid sequence position: 1192-1608, detection specificity: 98.3%.

NS3-21 recombinant antigen, mutations included on the NS3-20 sequence: C1305A, C1315A, C1318A, C1394A, C1400A, and C1454A, detection specificity: 99.8%.

NS3-30 recombinant antigen, corresponding to the HCV amino acid sequence position: 1192-1517, detection specificity: 98.4%.

NS3-31 recombinant antigen, mutations included on the NS3-30 sequence: C1305A, C1315A, C1318A, C1394A, C1400A, and C1454A, detection specificity: 99.9%.

NS3-40 recombinant antigen, corresponding to the HCV amino acid sequence position: 1192-1478, detection specificity: 98.0%.

NS3-41 recombinant antigen, mutations included on the NS3-40 sequence: C1305A, C1315A, C1318A, C1394A, C1400A, and C1454A, detection specificity: 99.8%.

NS3-50 recombinant antigen, corresponding to the HCV amino acid sequence position: 1230-1465, detection specificity: 98.1%.

NS3-51 recombinant antigen, mutations included on the NS3-50 sequence: C1305A, C1315A, C1318A, C1394A, C1400A, and C1454A, detection specificity: 99.8%.

NS3-52 recombinant antigen, mutations included on the NS3-50 sequence: C1454A, detection specificity: 98.7%.

NS3-53 recombinant antigen, mutations included on the NS3-50 sequence: C1318S, detection specificity: 98.2%.

NS3-54 recombinant antigen, mutations included on the NS3-50 sequence: C1400G, detection specificity: 98.6%.

NS3-55 recombinant antigen, mutations included on the NS3-50 sequence: C1305A, C1318A, and C1454A, detection specificity: 99.4%.

NS3-56 recombinant antigen, mutations included on the NS3-50 sequence: C1315S, C1394S, and C1454S, detection specificity: 98.3%.

NS3-57 recombinant antigen, mutations included on the NS3-50 sequence: C1315G, C1318G, and C1454G, detection specificity: 99.1%.

NS3-58 recombinant antigen, mutations included on the NS3-50 sequence: C1305A, C1315G, C1318A, C1394G,C1400A, and C1454A, detection specificity: 99.7%.

NS3-59 recombinant antigen, mutations included on the NS3-50 sequence: C1305G, C1315A, C1318A, C1394G,C1400A, and C1454A, detection specificity: 99.7%.

NS3-60 recombinant antigen, mutations included on the NS3-50 sequence: C1305A, C1315A, C1318G, C1394A,C1400A, and C1454A, detection specificity: 99.8%.

NS3-70 recombinant antigen, corresponding to the HCV amino acid sequence position: 1027-1657, detection specificity: 97.8%.

NS3-71 recombinant antigen, mutations included on theNS3-70 sequence: C1305A, C1315A, C1318G, C1394A,C1400A, and C1454A, detection specificity: 99.6%.

NS3-80 recombinant antigen, corresponding to HCV amino acid sequence position: 1380-1420, detection specificity: 98.3%.

NS3-81 recombinant antigen, mutations included on the NS3-80 sequence:C1394A and C1400A, detection specificity: 99.8%.

The corresponding sequences are shown as follows (the underlined position is the mutable position as described above):

NS3-10 (which is located in positions 1075-1657 of the HCV amino acid sequence, SEQ ID NO:1):

NGVCWTVYHGAGSKTLAGPKGPITQMYTNVDQDLVGWHRPPGARSLT
PCTCGSSDLYLVTRHADVIPVRRRGDSRGSLLSPRPVSYLKGSSGGPLLC
PFGHVAGIFRAAVCTRGVAKAVDFIPVETMETTMRSPVFTDNSSPPAVPQ
TFQVAHLHAPTGSGKSTKVPAAYAAQGYKVLVLNPSVAATLGFGAYMSKA
HGVDPNIRTGVRTITTGAPITYSTYGKFLADGGCSGGAYDIIICDECHST
DSTTILGIGTVLDQAETAGARLVVLATATPPGSVTVPHPNIEEVALSNTG
EIPFYGKAIPIEAIRGGRHLIFCHSKKKCDELAAKLSSLGLNAVAYYRGL
DVSVIPSSGDVVVVATDALMTGFTGDFDSVIDCNTCVTQTVDFSLDPTFT
IETTTVPQDAVSRSQRRGRTGRGREGIYRFVTPGERPSGMFDSSVLCECY
DAGCAWYELTPAETTVRLRAYLNTPGLPVCQDHLEFWEGVFTGLTHIDAH
FLSQTKQAGDNFPYLVAYQATVCAKAQAPPPSWDQMWKCLTRLKPTLQGP
TPLLYRLGAVQNEVTLTHPITKYIMTCMSADLEVVT

NS3-20 (which is located in positions 1192-1608 of the HCV amino acid sequence, SEQ ID NO:2):

AVDFIPVETMETTMRSPVFTDNSSPPAVPQTFQVAHLHAPTGSGKST
KVPAAYAAQGYKVLVLNPSVAATLGFGAYMSKAHGVDPNIRTGVRTITTG
APITYSTYGKFLADGGCSGGAYDIIICDECHSTDSTTILGIGTVLDQAET
AGARLVVLATATPPGSVTVPHPNIEEVALSNTGEIPFYGKAIPIEAIRGG
RHLIFCHSKKKCDELAAKLSSLGLNAVAYYRGLDVSVIPSSGDVVVVATD
ALMTGFTGDFDSVIDCNTAVFQTVDFSLDPTFTIETTTVPQDAVSRSQRR
GRTGRGREGIYRFVFPGERPSGMFDSSVLCECYDAGCAWYELTPAETTVR
LRAYLNTPGLPVCQDHLEFWEGVFTGLTHIDAHFLSQTKQAGDNFPYLVA
YQATVCAKAQAPPPSWDQMW

NS3-30 (which is located in positions 1192-1517 of the HCV amino acid sequence, SEQ ID NO:3):

AVDFIPVETMETTMRSPVFTDNSSPPAVPQTFQVAHLHAPTGSGKST
KVPAAYAAQGYKVLVLNPSVAATLGFGAYMSKAHGVDPNIRTGVRTITTG
APITYSTYGKFLADGGCSGGAYDIIICDECHSTDSTTILGIGTVLDQAET
AGARLVVLATATPPGSVTVPHPNIEEVALSNTGEIPFYGKAIPIEAIRGG
RHLIFCHSKKKCDELAAKLSSLGLNAVAYYRGLDVSVIPSSGDVVVVATD
ALMTGFTGDFDSVIDCNTAVTQTVDFSLDPTFTlETTTVPQDAVSRSQRR
GRTGRGREGlYRFVTPGERPSGMFDSSVL

NS3-40 (which is located in positions 1192-1478 of the HCV amino acid sequence, SEQ ID NO:4):

AVDFIPYETMETTMRSPVFTDNSSPPAYPQTFQVAHLHAPTGSGKST
KVPAAYAAQGYKVLYLNPSVAATLGFGAYMSKAHGVDPNIRTGVRTITTG
APITYSTYGKFLADGGCSGGAYDIIICDECHSTDSTTILGIGTVLDQAET
AGARLVVLATATPPGSVTVPHPNIEEVALSNTGEIPFYGKAIPIEAIRGG
RHLIFCHSKKKCDELAAKLSSLGLNAVAYYRGLDVSVIPSSGDVVVVATD
ALMTGFTGDFDSVIDCNTAVTQTVDFSLDPTFTlETTTVP

NS3-50 (which is located in positions 1230-1465 of the HCV amino acid sequence, SEQ ID NO:5):

APTGSGKSTKVPAAYAAQGYKVLVLNPSVAATLGFGAYMSKAHGVDP
NIRTGVRTITTGAPITYSTYGKFLADGGCSGGAYDIIIRDECHSTDSTTI
LGIGTVLDQAETAGARLVVLATATPPGSVTVPHPNIEEVALSNTGEIPFY
GKAIPIEAIRGGRHLIFCHSKKKCDELAAKLSSLGLNAVAYYRGLDVSVI
PSSGDVVVVATDALMTGFTGDFDSVIDCNTAVTOTVDFS

NS3-70 (which is located in positions 1027-1657 of the HCV amino acid sequence, SEQ ID NO:6):

APITAYSQQTRGLLGCIITSLTGRDKNQVEGEVQVVSTATQSFLATC
INGVCWTVYHGAGSKTLAGPKGPITQMYTNVDQDLVGWHRPPGARSLTPC
TCGSSDLYLVTRHADVIPVRRRGDSRGSLLSPRPVSYLKGSSGGPLLCPF
GHVAGIFRAAVCTRGVAKAVDFIPVETMETTMRSPVFTDNSSPPAVPQTF
QVAHLHAPTGSGKSTKVPAAYAAQGYKVLVLNPSVAATLGFGAYMSKAHG
VDPNIRTGVRTITTGAPITYSTYGKFLADGGCSGGAYDIIICDECHSTDS
TTILGIGTVLDOAETAGARLVVLATATPPGSVTVPHPNIEEVALSNTGEI
PFYGKAIPIEAIRGGRHLIFCHSKKKCDELAAKLSSLGLNAVAYYRGLDV
SVIPSSGDVVVVATDALMTGFTGDFDSVIDCNTCVTQTVDFSLDPTFTIE
TTTVPQDAVSRSQRRGRTGRGREGIYRFVTPGERPSGMFDSSVLCECYDA
GCAWYELTPAETTVRLRAYLNTPGLPVCQDHLEFWEGVFTGLTHIDAHFL
SOTKOAGDNFPYLVAYQATVCAKAQAPPPSWDQMWKCLTRLKPTLQGPTP
LLYRLGAVONEVTLTHPITKYIMTCMSADLEVVT

NS3-80 (which is located in positions 1380-1420 of the HCV amino acid sequence, SEQ ID NO:7): IPIEAIRGGRHLIFCHSKKKCDELAAKLSSLGLNAVAYYRG

### Embodiment 6

Partial NS3 region shown in Embodiment 5 was sequentially fused to an NS4 region and/or a core region, then the obtained HCV fusion antigen was prepared as a colloidal gold test strip, and 1000 clinically negative samples were detected to obtain a specificity detection result.

The sections included in the constructed recombinant antigen and detection specificity are described in detail below.

HCV-21 recombinant antigen, in which NS3-10 and Core-4 are fused sequentially from the N-terminal to the C-terminal, detection specificity: 98.20%.

HCV-22 recombinant antigen, in which NS3-11 and Core-4 are fused sequentially from the N-terminal to the C-terminal, detection specificity: 99.70%.

HCV-23 recombinant antigen, in which NS3-20 and NS4-4 are fused sequentially from the N-terminal to the C-terminal, detection specificity: 98.30%.

HCV-24 recombinant antigen, in which NS3-21 and NS4-4 are fused sequentially from the N-terminal to the C-terminal, detection specificity: 99.70%.

HCV-25 recombinant antigen, in which NS3-30, NS4-6 and Core-6 are fused sequentially from the N-terminal to the C-terminal, detection specificity: 98.20%.

HCV-26 recombinant antigen, in which NS3-31, NS4-6 and Core-6 are fused sequentially from the N-terminal to the C-terminal, detection specificity: 99.50%.

HCV-27 recombinant antigen, in which NS3-40, NS4-7 and Core-5 are fused sequentially from the N-terminal to the C-terminal, detection specificity: 98.40%.

HCV-28 recombinant antigen, in which NS3-41, NS4-7 and Core-5 are fused sequentially from the N-terminal to the C-terminal, detection specificity: 99.60%.

HCV-29 recombinant antigen, in which NS3-50 and Core-6 are fused sequentially from the N-terminal to the C-terminal, detection specificity: 98.10%.

HCV-30 recombinant antigen, in which NS3-51 and Core-6 are fused sequentially from the N-terminal to the C-terminal, detection specificity: 99.70%.

HCV-31 recombinant antigen, in which NS3-58, NS4-5 and Core-6 are fused sequentially from the N-terminal to the C-terminal, detection specificity: 99.50%.

HCV-32 recombinant antigen, in which NS3-59, NS4-5 and Core-6 are fused sequentially from the N-terminal to the C-terminal, detection specificity: 99.50%.

HCV-33 recombinant antigen, in which NS3-60, NS4-5 and Core-6 are fused sequentially from the N-terminal to the C-terminal, detection specificity: 99.60%.

### Embodiment 7

Mutation of either G/A/S in at least one site of 1305/1315/1318/1394/1400/1454 in the two NS3 regions in the HCV-5 recombinant HCV antigen, leaving the rest of the region unchanged, then the obtained HCV fusion antigen was prepared as acolloidal gold test strip, and 1000 clinically negative samples were detected to obtain a specificity detection result. HCV-5 recombinant HCV antigen is a fusion polypeptide fusing a first NS3 region, a second NS3 region, a first NS4 region, a second NS4 region, a third NS4 region and a CORE region from the N-terminal to the C-terminal, and the regions are respectively in order: NS3-3, NS3-7, NS4-3, NS4-4, NS4-5, and Core-6, without linkers.

Mutations, detection specificity and sensitivity included in the constructed recombinant antigen are described in detail below.

HCV-5 recombinant antigen, including NS3-3 and NS3-7, without mutations, detection specificity: 98.3%, and sensitivity: 98%.

HCV-51 recombinant antigen, including NS3-3 withC1318S; NS3-7 withC1400S, detection specificity: 98.4%, and sensitivity: 98%.

HCV-52 recombinant antigen, including NS3-3 with C1454A; NS3-7 with C1394A, detection specificity: 99.0%, and sensitivity: 98%.

HCV-53 recombinant antigen, including NS3-3 withC1315G; NS3-7 with C1394G, detection specificity: 98.9%, and sensitivity: 98%.

HCV-54 recombinant antigen, including NS3-3with C1305S and C1318S; NS3-7 with C1394S and C1400S, detection specificity: 98.5%, and sensitivity: 98%.

HCV-55 recombinant antigen, including NS3-3 with C1315G and C1318A; NS3-7 with C1394A and C1400G, detection specificity: 99.3%, and sensitivity: 98%.

HCV-56 recombinant antigen, including NS3-3 with C1315G, C1318A, C1394A and C1400A; NS3-7 with C1394G and C1400A, detection specificity: 99.6%, and sensitivity: 98%.

HCV-57 recombinant antigen, including NS3-3 with C1305A, C1315A, C1318A, C1394G and C1400A; NS3-7 with C1394G and C1400A, detection specificity: 99.8%, and sensitivity: 98%.

HCV-58 recombinant antigen, including NS3-3 with C1305A, C1315A, C1318A, C1394A, C1400A and C1454A; NS3-7 with C1394A and C1400A, detection specificity: 99.9%, and sensitivity: 98%.

Note: specificity is calculated as the detection rate of 1000 negative samples, and sensitivity is calculated as the detection rate of 100 RIBA-confirmed positive samples.

The sensitivity is obtained by detecting the 100 RIBA-confirmed positive samples by using the recombinant antigen constructed above, and is specifically calculated as follows:

$$\text{Sensitivity} = \left[\text{TP} / \left(\text{TP+FN}\right)\right] \times 100 \%$$

Where TP is the number of samples with positive (true positive) detection results, and FN is the number of samples with negative (false negative) detection results.

With regard to this embodiment, the sensitivity is the number of samples with positive detection results divided by 100 and multiplied by 100%.

Corresponding sequences in Embodiments 2 and 3 are shown as follows:

NS3-3 (which is located in positions 1230-1465 of the HCV amino acid sequence, SEQ ID NO:8):

APTGSGKSTKVPAAYAAQGYKVLVLNPSVAATLGFGAYMSKAHGVDP
NIRTGVRTITTGAPITYSTYGKFLADGGCSGGAYDIIICDECHSTDSTTI
LGIGTVLDQAETAGARLV11LATATPPGSVTVPHPNIEEVALSNTGEIPF
YGKAIPIEAIRGGRHLIFCHSKKKCDELAAKLSSLGLNAVAYYRGLDVSV
IPSSGDVVVVATDALMTGFTGDFDSVIDCNTCVTQTVDFS

NS3-7 (which is located in positions 1377-1443 of the HCV amino acid sequence, SEQ ID NO:9):

GKAIPLEVIKGGRHLIFCHSKKKCDELAAKLVALGINAVAYYRGLDV
SVIPTSGDVVVVVATDALMTG

NS4-3 (which is located in positions 1691-1749 of the HCV amino acid sequence, SEQ ID NO:11):

KPAIIPDREVLYREFDEMEECSQHLPYIEQGMMLAEQFKQKALGLLQ
TATKQAEAAAPV

NS4-4 (which is located in positions 1695-1741 of the HCV amino acid sequence, SEQ ID NO:12):

GGKPALVPDKEVYLYQQYDEMEECSQAAPYIEQAQVIAHQFKEKVLG
L

NS4-5 (which is located in positions 1693-1740 of the HCV amino acid sequence, SEQ ID NO:13):

NDQVVVTPDKEILYEAFDEMEECASKAALIEEGQRMAEMLKSKIQGL
L

NS4-6 (which is located in positions 1698-1931 of the HCV amino acid sequence, SEQ ID NO:14):

REVLYREFDEMEECSQHLPYlEQGMMLAEQFKQKALGLLQTASRQAE
ATAPVVQSKWQSLETFWAKHMWNFISGIQYLAGLSTLPGNPAIASLMAFT
AAVTSPFSTQQTLLFNILGGVWAAQLAAPSAATSFVGAGIAGAAIGSVGL
GKVLVDILAGYGAGVAGALVAFKVMSGEVPTTEDLVNLLPAILSPGALVV
GVVCAAILRRHVGPGEGAVQWMNRLIAFASRGNHVSP

NS4-7 (which is located in positions 1691-1799 of the HCV amino acid sequence, SEQ ID NO:15):

KPAIIPDREVLYREFDEMEECSQHLPYIEQGMMLAEQFKQKALGLLQ
TASRQAEATAPVVQSKWQSLETFWAKHMWNFISGlQYLAGLSTLPGNPAI
ASLMAFTAAVTS

Core-4 (which is located in positions 1-80 of the HCV amino acid sequence, SEQ ID NO:16):

MSTNPKPQRKTKRNTDRRPQDVKFPGGGQIVGGVYLLPRRGPRLGVR
ATRKTSERSQPRGRRQPIPKARRPEGRTWAQPG

Core-5 (which is located in positions 1-53 of the HCV amino acid sequence, SEQ ID NO:17):

MSTNPKPQRKTKRNTDRRPQDVKFPGGGQIVGGVYLLPRRGPRLGVR
ATRKTS

Core-6 (which is located in positions 1-48 of the HCV amino acid sequence, SEQ ID NO:18):

MSTNPKPQRKTKRNTDRRPQDVKFPGGGQIVGGVYLLPRRGPRLGVR
A.

The above are only the preferred implementations of the application and are not intended to limit the disclosure. For those skilled in the art, the application may have various modifications and variations. Any modifications, equivalent replacements, improvements and the like made within the spirit and principle of the disclosure shall fall within the scope of protection of the disclosure.

### INDUSTRIAL APPLICABILITY

The method and/or products in the application solve one or more problems of low sensitivity and low specificity. Therefore, compared with existing methods, in the application, the sensitivity and/or specificity of HCV antibody detection are/is significantly improved.

What is claimed is:

1. An HCV recombinant antigen, comprising at least one NS3 antigen, wherein at least one cysteine in the NS3 antigen is mutated into G or A; and the mutation site is located between the HCV amino acid positions 1192 and 1517.

2. The HCV recombinant antigen according to claim 1, wherein the HCV recombinant antigen comprising at least one NS3 antigen, wherein at least one cysteine of the NS3 antigen is mutated into G or A and preferably mutated into A at position 1305/1315/1318/1394/1400/1454 of a corresponding HCV amino acid sequence.

3. The HCV recombinant antigen according to claim 1, wherein the specificity of the HCV recombinant antigen for detecting an HCV antibody is not less than 98.0%, optionally, not less than 98.2%, not less than 99.0%, not less than 99.6%, not less than 99.8% and not less than 99.9%.

4. The HCV recombinant antigen according to claim 1, wherein the NS3 antigen is mutated from a NS3 polypeptide selected from positions 1027-1657 of the HCV amino acid sequence.
optionally, the length of the polypeptide is 41-631 amino acids, optionally, 41, 50, 60, 70, 80, 90, 100, 150, 200, 250, 300, 350, 400, 450, 500, 550 or 600 amino acids;
optionally, the NS3 polypeptide comprises positions 1027-1657, 1380-1420, 1075-1657, 1230-1465, 1192-1478, 1377-1443, 1192-1608 or 1192-1517 of the HCV amino acid sequence;
optionally, the NS3 polypeptide comprises sequences selected from SEQ ID NOs:1-9.

5. The HCV recombinant antigen according to claim 1, wherein the HCV recombinant antigen further comprises an another HCV antigen,
optionally, another one or more, at least one, two, three or more NS3 antigens;
another one or more, at least one, two, three or more NS4 antigens; another one or more, at least one, two, three or more NS5 antigens; and/or another one or more, at least one, two, three or more core antigens.

6. The HCV recombinant antigen according to claim 5, wherein the NS3 antigen, the NS4 antigen, the NS5 antigen and/or the core antigen are directly connected or connected optionally by means of one or more linkers, the linker is (G)n, (GS)n, (SG)n, (GGGS)n or (GGGGS)n, wherein n is one, two, three, four, five, six, seven, eight, nine, ten or a larger integer.

7. The HCV recombinant antigen according to claim 1, wherein the HCV recombinant antigen comprises N- and/or C-terminal modifications;
optionally, the modifications comprise histidine tags, biotinylation and/or detectable marker modifications.

8. The HCV recombinant antigen according to claim 1, wherein HCV comprises HCV genotype 1a, 1b, 2a, 2b, 2c, 2d, 3a, 3b, 3c, 3d, 3e, 3f, 4a, 4b, 4c, 4d, 4e, 4f, 4g, 4h, 4i, 4j, 5a or 6a.

9. The HCV recombinant antigen according to claim 2, wherein the NS3 antigen has cysteine mutations at positions 1305, 1315, 1318, 1394, 1400 and 1454 of the corresponding HCV amino acid sequence; the cysteine mutation at each position independently selects the mutation from cysteine to G or the mutation from the cysteine to A; and preferably, the cysteine mutation at each of the position is the mutation from the cysteine to A.

10. A nucleic acid encoding the HCV recombinant antigen according to claim 1, or an expression vector comprising the nucleic acid, or a host cell comprising the expression vector.

11. A conjugate, comprising the HCV recombinant antigen according to claim 1.

12. The conjugate according to claim 11, further comprising a solid support, a detectable marker or a binding partner which is conjugated with the HCV recombinant antigen;
optionally, the HCV recombinant antigen in the conjugate is directly or indirectly conjugated;
optionally, the solid support comprises magnetic particles, a microtiter plate or a cellulose membrane;
optionally, the detectable marker is a metal particle, a fluorescent marker, a chromophore marker, an electron-dense marker, a chemiluminescent marker, a
radioactive marker, or an enzyme marker;
optionally, the detectable marker is colloidal gold, radio isotope, fluorophores, a spin marker, or a bacteriophage marker;
optionally, the detectable marker is rhodamine, fluorescein, acridinium ester, luciferase, horseradish peroxidase, alkaline phosphatase, β-galactosidase, glucoamylase, lysozyme, carbohydrate oxidase, or a glucose-6-phosphate dehydrogenase marker;
optionally, the carbohydrate oxidase comprises glucose oxidase or galactose oxidase;
optionally, the binding partner comprises biotin, or avidin;
optionally, the avidin comprises streptavidin.

13. A kit, comprising the HCV recombinant antigen according to claim 1, or the conjugate comprising the HCV recombinant antigen according to claim 1.

14. Applications of the HCV recombinant antigen according to claim 1 or the conjugate comprising the HCV recombinant antigen according to claim 1 in preparation of a kit for detecting a Hepatitis C Virus antibody or antigen from a sample of a subject.

15. Applications of the HCV recombinant antigen according to claim 1 or the conjugate comprising the HCV recombinant antigen according to claim 1 for detecting a Hepatitis C Virus antibody or antigen from a sample of a subject.

16. A method for detection a Hepatitis C Virus antibody or antigen in a sample of a subject, comprising contacting the HCV recombinant antigen according to claim 1 or the conjugate comprising the HCV recombinant antigen according to claim 1 with a sample of a subject.

17. A method for diagnosing hepatitis C, comprising:
using the HCV recombinant antigen according to claim 1 or the conjugate comprising the HCV recombinant antigen according to claim 1 to detect a Hepatitis C Virus antibody or antigen from a sample of a subject; and analyzing the detection result.
